(19) United States
(12) Patent Application Publication
(10) Pub. No.: US 2013/0184294 A1
(43) Pub. Date: Jul. 18, 2013

(54) Pyrimidones for Treatment of Potassium Channel Related Diseases

(71) Applicants: Isao Sakurada, Gotemb (JP); Lei Zhang, Auburndale, MA (US)

(72) Inventors: Isao Sakurada, Gotemb (JP); Lei Zhang, Auburndale, MA (US)

(73) Assignee: Pfizer Inc., Groton, CT (US)

(21) Appl. No.: 13/822,669

(22) PCT Filed: Sep. 7, 2011
(86) PCT No.: PCT/IB11/53917

§ 371 Date: Mar. 13, 2013

Related U.S. Application Data
(60) Provisional application No. 61/385,138, filed on Sep. 21, 2010.

(51) Int. Cl. C07D 471/04; A61K 45/06; A61K 31/519

(52) U.S. Cl. CPC C07D 471/04; A61K 31/519; A61K 45/06
USPC 514/259.4; 544/282

(57) ABSTRACT

The present invention relates to compounds of Formula I as described herein or a pharmaceutically acceptable salt thereof, pharmaceutical composition comprising a compound of Formula I or a pharmaceutically acceptable salt thereof and methods of treating a disease, disorder, or condition of the central nervous system, including bipolar disorder, depressive disorders, anxiety disorders, cognitive disorders, pain disorders, urogenital disorders, and epilepsy, among the other diseases, disorders or conditions discussed herein as mono-therapy or in combination with another active pharmaceutical ingredient.

### FIELD OF THE INVENTION

The present invention relates to pyrimidones and to pharmaceutical compositions containing them and to their use in the treatment of central nervous system disorders, including bipolar disorder, depressive disorders, anxiety disorders, cognitive disorders, pain disorders, urogenital disorders, epilepsy and other disorders in mammals, including humans. The present invention relates to compounds, which are openers of voltage dependent potassium channels of the Kv7.2/7.3 or KCNQ2/3 subtype. The compounds are useful in the treatment of disorders and diseases affected by dampening the excitability of tissues expressing and responsive to the Kv7 family (Kv7.2, 7.3, 7.4, 7.5 subtypes) of voltage dependent potassium channels. These compounds have been shown to facilitate the opening of the Kv7.2-5 voltage dependent potassium channels

### BACKGROUND OF THE INVENTION

Kv7.2/3 channels are voltage-gated potassium channels that modulate neuronal excitability in the central and peripheral nervous systems. Blockade of Kv7.2./7.3.channels, for example, by acetylcholine, increases neuronal excitability, whereas channel opening decreases it. Kv7 channels are expressed as homo or heterotetramers, composed of different subunit combinations. A deficiency in these channels is the underlying cause of a rare form of neonatal epilepsy, and polymorphisms in the Kv7.3 gene are associated with bipolar disorder based on linkage studies. The known Kv7 openers flupirtine (2-amino-6-[[(4-fluorophenyl)methyl]amino]-3-pyridinyl]-carbamic acid ethyl ester) and retigabine (N-(2-amino-4-(4-fluorobenzylamino)-phenyl)carbamic acid ethyl ester)) have shown numerous clinical applications, including, inter alia, epilepsy, pain, and cognitive function.

The present invention relates to pyrimidone compounds of Formula I that exhibit activity as Kv7.2-5 channel openers.

### SUMMARY OF THE INVENTION

The present invention relates to compounds of Formula I as described below, or a pharmaceutically acceptable salt thereof, or a stereoisomer of the compound of Formula I or a pharmaceutically acceptable salt thereof.

This invention also relates to a pharmaceutical composition comprising a compound of Formula I, or a pharmaceutically acceptable salt thereof, or a stereoisomer of the compound of Formula I or a pharmaceutically acceptable salt thereof, and at least one pharmaceutically acceptable excipient.

This invention also is directed to a method of treating, or manufacture of a medicament to treat, a disease, disorder or condition of the central nervous system, including bipolar disorder, depressive disorders, anxiety disorders, cognitive disorders, pain disorders, urogenital disorders, and epilepsy, among the other diseases, disorders, or conditions discussed herein as mono-therapy or in combination with one or more pharmaceutical active ingredient.

### DETAILED DESCRIPTION OF THE INVENTION

An embodiment of the invention is a compound of Formula I

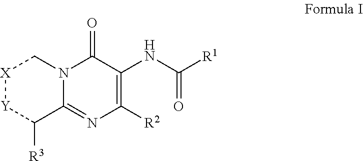

wherein:

R¹ is alkyl or cycloalkyl, wherein alkyl or cycloalkyl may be substituted with one or more halogen, alkoxy, aryl, or aryloxy;

R² is cycloalkyl or NR⁴R⁵;

R³ is H, halogen, alkyl, or alkoxy, wherein any alkyl may be substituted with one or more halogen atoms;

R⁴ and R⁵ are independently selected from H, C₁₋₆alkyl, C₃₋₆cycloalkyl, or —C₁₋₆alkyl-C₃₋₆cycloalkyl, wherein each alkyl or each cycloalkyl may be substituted with one or more halogen atoms, provided that both R⁴ and R⁵ are not H simultaneously;

or R⁴ and R⁵, together with the N atom to which they are attached, form a heterocycloalkyl;

—X—Y— is ═CH—CH═, —CH₂—CH₂—, or —CH₂—; or a pharmaceutically acceptable salt thereof.

This invention also relates to compounds of Formula I wherein R¹ is C₅ to C₆ alkyl, for example —CH₂C(CH₃)₃.

This invention also relates to compounds of Formula I wherein R² is NR⁴R⁵, and where one of R⁴ or R⁵ is H and the other is C₃₋₆alkyl, C₃₋₆cycloalkyl, or —C₁₋₃alkyl-C₃₋₆cycloalkyl. For example, C₃₋₆alkyl, C₃₋₆ cycloalkyl, or —C₁₋₃alkyl-C₃₋₆cycloalkyl include isopropyl, isobutyl, 1-cyclopropylethyl, cyclobutyl or cyclopentyl. Alternatively, the invention relates to compounds of Formula I wherein R² is cyclopropyl.

This invention also relates to compounds of Formula I wherein R³ is halogen, alkyl, or alkoxy, wherein any alkyl may be substituted with one or more halogen atoms giving haloalkyl and haloalkoxy, respectively. Furthermore, the invention relates to compounds of Formula I wherein R³ is halogen or alkyl, wherein alkyl may be substituted with one or more halogen atoms. Examples for R³ include chloro, methyl, trifluoromethyl and 1,1-difluoroethoxy.

The invention also relates to compounds of Formula I, wherein —X—Y— is ═CH—CH═ or —CH₂—CH₂— such that when they are taken together with the pyrimidone moiety create a bicyclic moiety that is 4-oxo-4H-pyrido[1,2-a]pyrimidin-3-yl or 4-oxo-6,7,8,9-tetrahydro-4H-pyrido[1,2-a]pyrimidin-3-yl.

It is understood that descriptions of any one substituent, such as R¹, may be combined with descriptions of any other substituents, such as R², such that each and every combination of the first substituent and the second substituent is provided herein the same as if each combination were specifically and individually listed. For example, in one variation, R¹ is —CH₂C(CH₃)₃; and R² is NR⁴R⁵, wherein R⁴ is H and R⁵ is isopropyl, isobutyl, 1-cyclopropylethyl, cyclobutyl or cyclopentyl.

The invention further concerns each example provided herein as restated independently by name here. The invention could include all compounds, or, alternatively, include a smaller group. For example, the invention could include all forms of Examples 1 to 11 or could include, e.g., any one example alone or named together with fewer than all examples.

Also desired are novel compounds with improved properties relative to known compounds, which are openers of the KCNQ family potassium channels, such as retigabine. Improvement of one or more of the following parameters is desired: half-life, clearance, and selectivity, interactions with other medications, bioavailability, potency, formulability, chemical stability, metabolic stability, membrane permeability, solubility and therapeutic index. The improvement of such parameters may lead to improvements such as: an improved dosing regime by reducing the number of required doses a day, ease of administration to patients on multiple medications, reduced side effects, greater therapeutic index, improved tolerability or improved compliance.

### ABBREVIATIONS AND DEFINITIONS

Unless otherwise indicated, as used herein, the terms “halogen” and “halo” include fluoro, chloro, bromo, and iodo.

Unless otherwise indicated, as used herein, the term “alkyl” includes saturated monovalent hydrocarbon radicals containing from one to ten carbon atoms unless otherwise specified and having straight or branched moieties. Examples of alkyl groups include methyl, ethyl, n-propyl, isopropyl, tert-butyl, and CH₂C(CH₃)₃

Unless otherwise indicated, as used herein, the term “cycloalkyl” includes saturated monovalent hydrocarbon cyclic moieties. Examples of cycloalkyl groups include cyclopropyl, cyclobutyl, cyclopentyl, and cyclohexyl.

The term “alkyl-cycloalkyl” refers to an alkyl substituent, as defined herein, substituted by a cycloalkyl substituent, as defined herein.

A prefix attached to a multi moiety substituent only applies to the first moiety. To illustrate, the term “alkylcycloalkyl” contains two moieties: alkyl and cycloalkyl. Thus, a C₁₋₆ prefix on C₁₋₆alkylcycloalkyl means that the alkyl moiety of the alkylcycloalkyl contains from 1 to 6 carbon atoms; the C₁₋₆ prefix does not describe the cycloalkyl moiety.

Unless otherwise indicated, as used herein, the term “haloalkyl” includes an alkyl moiety substituted by at least one halogen atom selected from fluorine (fluoro, F), chlorine (chloro, Cl), bromine (bromo, Br), or iodine (iodo, I). The number of halogens for substitution will depend on valency of the alkyl moiety. For example, and not by way of limitation, examples include for methyl: CH₂F, CHF₂, and CF₃.

Unless otherwise indicated, as used herein, the term “haloalkoxyl” includes an alkyloxy moiety wherein the alkyl is substituted by at least one halogen atom selected from fluorine (fluoro, F), chlorine (chloro, Cl), bromine (bromo, Br), or iodine (iodo, I). The number of halogens for substitution will depend on valency of the alkyl moiety. For example, and not by way of limitation, a non-exhaustive list of examples for haloethoxy include: O—CH₂—CH₂F, O—CH₂—CHF₂, O—CH₂—CF₃, O—CF₂—CH₃, O—CF₂—CH₂F, O—CF₂—CHF₂, and O—CF₂—CF₃.

Unless otherwise indicated, as used herein, the term heterocycloalkyl means a mono-cycloalkyl moiety of 4 to 10 carbons where at least one carbon atom has been replaced with a heteroatom selected from nitrogen, oxygen, or sulfur and where not all of the carbon atoms must be part of the ring. Examples of such heterocycloalkyl rings include azetidinyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydro-thiadiazinyl, morpholinyl, oxetanyl, methyloxetanyl, tetrahydrodiazinyl, oxazinyl, oxathiazinyl, indolinyl, isoindolinyl, quinuclidinyl, chromanyl, isochromanyl, benzoxazinyl, and the like.

The term “aryl” refers to an aromatic substituent containing one ring or two fused rings. The aryl substituent may have six to eighteen carbon atoms. As an example, the aryl substituent may have six to fourteen carbon atoms. The term “aryl” may refer to substituents such as phenyl, naphthyl and anthracenyl. The term “aryl” also includes substituents such as phenyl, naphthyl and anthracenyl that are fused to a C₄-C₁₀ carbocyclic ring, such as a C₅- or a C₆-carbocyclic ring, or to a 4-10-membered heterocyclic ring, wherein a group having such a fused aryl group as a substituent is bound to an aromatic carbon of the aryl group. When such a fused aryl group is substituted with one or more substituents, the one or more substituents, unless otherwise specified, are each bound to an aromatic carbon of the fused aryl group. Examples of aryl groups include accordingly phenyl, naphthalenyl, tetrahydronaphthalenyl (also known as “tetralinyl”), indenyl, isoindenyl, indanyl, anthracenyl, phenanthrenyl, and benzonaphthenyl (also known as “phenalenyl”).

Unless otherwise indicated, as used herein, the term “alkoxy” and the term “aryloxy” mean “alkyl-O—” and “aryl-O—”, respectively, wherein “alkyl” and “aryl” are as defined herein. Examples of “alkoxy” groups include methoxy, ethoxy, propoxy, butoxy, pentoxy, allyloxy, and O-cycloalkyl. An example of aryloxy includes —O-phenyl.

Unless otherwise indicated, the term “one or more” substituents, or “at least one” substituent as used herein, refers to from one to the maximum number of substituents possible based on the number of available bonding sites. Examples of “one or more” or “at least one” substituent include 1 to 3 substituents on a terminal methyl, e.g., 3 available bonding sites where the fourth bonding site is from the methyl to molecule for which carbon of the methyl is a terminal atom.

The following abbreviations are used herein:

EtOAc: Ethyl acetate

HPLC: High pressure liquid chromatography

LCMS: Liquid chromatography-mass spectrometry

MS: Mass spectrometry

RT: Room temperature

Non-limiting, specific embodiments of the present invention are shown in the Examples below.

Compounds of Formula I may have optical centers and therefore may occur in different enantiomeric and diastereomeric configurations. The present invention includes all enantiomers, diastereomers, and other stereoisomers of such compounds of Formula I, as well as racemic compounds and racemic mixtures and other mixtures of stereoisomers thereof.

Compounds of Formula I include all forms of the compound of Formula I, including solvates including hydrates when the solvent is water, isomers, crystalline and non-crystalline forms, isomorphs, polymorphs, metabolites, and prodrugs thereof. For example, the compounds of Formula I, or a pharmaceutically acceptable salt thereof, may exist in unsolvated and solvated forms. When the solvent, including water, is tightly bound, the complex will have a well-defined stoichiometry independent of humidity. When, however, the solvent is weakly bound, as in channel solvates and hygroscopic compounds, the solvent content will be dependent on humidity and drying conditions. In such cases, non-stoichiometry will be the norm; this routinely occurs when one is isolating final compound from a reaction mixture, and upon drying, residual solvent, including water, remains present. Failure to provide peaks in reporting spectral data of said solvates, including hydrates, is normal because one of ordinary skill in the art would expect such solvates to be present and normally reports only peaks used to identify structure and normally does not report solvent peaks. Therefore, when referring to a compound of Formula I or a compound of the invention, it is meant to include residual solvent including water, isomers, crystalline and non-crystalline forms, isomorphs, enantiomers, diastereomers, other stereoisomers polymorphs, metabolites, and prodrugs of the compounds of Formula I and also of the corresponding pharmaceutically acceptable salts thereof.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4th Edition (Edward Arnold, 1970).

The compounds of the invention include compounds of Formula I as hereinbefore defined, including all polymorphs and crystal habits thereof, prodrugs and isomers thereof (including optical, geometric and tautomeric isomers) as hereinafter defined and isotopically-labeled compounds of Formula I.

As indicated, so-called ‘prodrugs’ of the compounds of Formula I are also within the scope of the invention. Thus, certain derivatives of compounds of Formula I which may have little or no pharmacological activity themselves can, when administered into or onto the body, be converted into compounds of Formula I having the desired activity, for example, by hydrolytic cleavage. Such derivatives are referred to as ‘prodrugs’. Further information on the use of prodrugs may be found, for example, in Pro-drugs as Novel Delivery Systems, Vol. 14, ACS Symposium Series (T. Higuchi and W. Stella). See also Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of Formula I with certain moieties known to those skilled in the art as ‘pro-moieties’ as described, for example, in Design of Prodrugs by H. Bundgaard (Elsevier, 1985).

Some examples of prodrugs in accordance with the invention include
(i) where the compound of Formula I contains a carboxylic acid functionality (—COOH), an ester thereof, for example, a compound wherein the hydrogen of the carboxylic acid functionality of the compound of Formula I is replaced by (C₁-C₈)alkyl;
(ii) where the compound of Formula I contains an alcohol functionality (—OH), an ether thereof, for example, a compound wherein the hydrogen of the alcohol functionality of the compound of Formula I is replaced by (C₁-C₆)alkanoyloxymethyl; and
(iii) where the compound of Formula I contains a primary or secondary amino functionality (—NH₂ or —NHR where R is not H), an amide thereof, for example, a compound wherein, as the case may be, one or both hydrogens of the amino functionality of the compound of Formula I is/are replaced by (C₁-C₁₀)alkanoyl.

Further examples of replacement groups in accordance with the foregoing examples and examples of other prodrug types may be found in the aforementioned references.

Moreover, certain compounds of Formula I may themselves act as prodrugs of other compounds of Formula I.

Also included within the scope of the invention are metabolites of compounds of Formula I, that is, compounds formed in vivo upon administration of the drug. Some examples of metabolites in accordance with the invention include
(i) where the compound of Formula I contains a methyl group, a hydroxymethyl derivative thereof (—CH₃→—CH₂OH):
(ii) where the compound of Formula I contains an alkoxy group, a hydroxy derivative thereof (—OR→—OH);
(iii) where the compound of Formula I contains a tertiary amino group, a secondary amino derivative thereof (—NR¹R²→—NHR¹ or —NHR²);
(iv) where the compound of Formula I contains a secondary amino group, a primary derivative thereof (—NHR¹→—NH₂);
(v) where the compound of Formula I contains a phenyl moiety, a phenol derivative thereof (-Ph→-PhOH); and
(vi) where the compound of Formula I contains an amide group, a carboxylic acid derivative thereof (—CONH₂→COOH).

Compounds of Formula I containing one or more asymmetric carbon atoms can exist as two or more stereoisomers. Where a compound of Formula I contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of Formula I containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of Formula I, including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate or racemic mixture (or a racemic precursor) may be reacted with a suitable optically active compound, for example, an alcohol, or, in the case where the compound of Formula I contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art; see, e.g., Stereochemistry of Organic Compounds by E. L. Eliel and S. H. Wilen (Wiley, 1994).

The present invention includes all pharmaceutically acceptable isotopically-labeled compounds of Formula I wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H; carbon, such as ¹¹C, ¹³C, and ¹⁴C; chlorine, such as ³⁶Cl; fluorine, such as ¹⁸F; iodine, such as ¹²³I and ¹²⁵I; nitrogen, such as ¹³N and ¹⁵N; oxygen, such as ¹⁵O, ¹⁷O, and ¹⁸O; phosphorus, such as ³²P; and sulfur, such as ³⁵S.

Certain isotopically-labeled compounds of Formula I, for example, those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection.

Substitution with heavier isotopes such as deuterium (²H) may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of Formula I can generally be prepared by conventional techniques known to those skilled in the art or by processes analogous to those described in the accompanying Examples and Preparations using an appropriate isotopically-labeled reagent in place of the non-labeled reagent previously employed.

When preparing compounds of Formula I in accordance with the invention, it is open to a person skilled in the art to routinely select the form of compound of Formula I that provides the best combination of features for this purpose. Such features include the melting point, solubility, processability and yield of the intermediate form and the resulting ease with which the product may be purified on isolation.

As used herein, the term “treating” refers to reversing, alleviating or inhibiting the progress of a disease, disorder or condition, or one or more symptoms of such disease, disorder or condition, to which such term applies. As used herein, “treating” may also refer to decreasing the probability or incidence of the occurrence of a disease, disorder or condition in a mammal as compared to an untreated control population, or as compared to the same mammal prior to treatment. For example, as used herein, “treating” may refer to preventing a disease, disorder or condition, and may include delaying or preventing the onset of a disease, disorder or condition, or delaying or preventing the symptoms associated with a disease, disorder or condition. As used herein, “treating” may also refer to reducing the severity of a disease, disorder or condition or symptoms associated with such disease, disorder or condition prior to a mammal's affliction with the disease, disorder or condition. Such prevention or reduction of the severity of a disease, disorder or condition prior to affliction relates to the administration of the composition of the present invention, as described herein, to a subject that is not at the time of administration afflicted with the disease, disorder or condition. As used herein “treating” may also refer to preventing the recurrence of a disease, disorder or condition or of one or more symptoms associated with such disease, disorder or condition. The terms “treatment” and “therapeutically,” as used herein, refer to the act of treating, as “treating” is defined above.

The compounds of the present invention dampen neuronal excitability and therefore are of value in the treatment of a wide variety of clinical diseases, disorders, or conditions that are characterized by the dysregulation of neuronal excitability in mammalian subjects, especially humans. Such diseases, disorders, or conditions include the various types of epilepsy, pain disorders, (e.g. diabetic neuropathy, fibromyalgia, migraine, post-herpetic neuralgia) and bipolar disorder (e.g., bipolar types I & II and rapid cycling). Compounds of the present invention are useful in the treatment of, for example, anxiety disorders including generalized anxiety disorder, panic disorder, PTSD, and social anxiety disorder; mood adjustment disorders including depressed mood, mixed anxiety and depressed mood, disturbance of conduct, and mixed disturbance of conduct and depressed mood; attention adjustment disorders including ADHD, attention deficit disorders or other cognitive disorders due to general medical conditions; psychotic disorders including schizoaffective disorders and schizophrenia; and sleep disorders including narcolepsy and enuresis.

Examples of the diseases, disorders or conditions which may be treated by the compound, composition and method of this invention are also as follows: depression, including depression in cancer patients, depression in Parkinson's patients, post-myocardial Infarction depression, depression in patients with human immunodeficiency virus (HIV), Subsyndromal Symptomatic depression, depression in infertile women, pediatric depression, major depression, single episode depression, recurrent depression, child abuse induced depression, postpartum depression, DSM-IV major depression, treatment-refractory major depression, severe depression, psychotic depression, post-stroke depression, neuropathic pain, manic depressive illness, including manic depressive illness with mixed episodes and manic depressive illness with depressive episodes, seasonal affective disorder, bipolar depression BP I, bipolar depression BP II, or major depression with dysthymia; dysthymia; phobias, including agoraphobia, social phobia or simple phobias; eating disorders, including anorexia nervosa or bulimia nervosa; chemical dependencies, including addictions to alcohol, cocaine, amphetamine and other psychostimulants, morphine, heroin and other opioid agonists, Phenobarbital and other barbiturates, nicotine, diazepam, benzodiazepines and other psychoactive substances; Parkinson's disease, including dementia in Parkinson's disease, neuroleptic-induced parkinsonism or tardive dyskinesias; headache, including headache associated with vascular disorders; withdrawal syndrome; age-associated learning and mental disorders; apathy; bipolar disorder; chronic fatigue syndrome; chronic or acute stress; conduct disorder; cyclothymic disorder; somatoform disorders such as somatization disorder, conversion disorder, pain disorder, hypochondriasis, body dysmorphic disorder, undifferentiated disorder, and somatoform NOS; incontinence; inhalation disorders; intoxication disorders; mania; oppositional defiant disorder; peripheral neuropathy; post-traumatic stress disorder; late luteal phase dysphoric disorder; specific developmental disorders; SSRI “poop out” syndrome, or a patient's failure to maintain a satisfactory response to SSRI therapy after an initial period of satisfactory response; and tic disorders including Tourette's disease.

Compounds of the present invention are also useful for the treatment of epilepsy, pain, and cognitive function. See, e.g., Cooper E C, Jan L Y. Arch Neurol. 2003 April, 60(4):496-500. Furthermore, the compounds of the present invention are useful for the treatment of central nervous system disorders, including bipolar disorder, depressive disorders, anxiety disorders, cognitive disorders, pain disorders, urogenital disorders, and epilepsy.

Compounds of Formula I and pharmaceutically acceptable salts thereof, in combination with other active pharmaceutical active ingredients are useful to treat various diseases or disorders. For example, compounds of Formula I and pharmaceutically acceptable salts thereof may be combined with anti-convulsants (e.g., acetazolamide, carbamazepine, clobazam, clonazepam, diazepam, divalproex sodium, ethosuximide, ethotoin, felbamate, fosphenyloin, gabapentin, lamotrigine, levetiracetam, mephenyloin, metharbital, methsuximide, methazolamide, oxcarbazepine, phenobarbital, phenyloin, phensuximide, pregabalin, primidone, sodium valproate, stiripentol, tiagabine, topiramate, trimethadione, valproic acid, vigabatrin, zonisamide) to treat disorders the treatment of which is facilitated by decreased neurotransmission such as epilepsy (generalized or partial seizure disorder), nonepileptic seizures such as febrile seizures, symptomatic seizures and psychogenic seizures. Because the anti-convulsants and the compounds of Formula I have differing mechanisms, the compounds of Formula I provides additional control of dysregulated excitability that the current therapies do not provide. This would be the case especially with older antiepileptics such as phenyloin and carbemazepine. See Azar N J, Abou-Khalil B W. Semin Neurol., 2008 July; 28(3):305-16.

Compounds of Formula I and pharmaceutically acceptable salts thereof, in combination with mood stabilizing compounds (e.g lithium carbonate, valproic acid, lamotrigine, carbamazepine oxcarbazepine and atypical antipsychotics [e.g., clozapine, quetiapine, olanzapine, ziprasidone]) are useful psychotherapeutics and may be used in the treatment of bipolar disorders (depressive episode, manic episode, hypomanic episode, mixed affective episode) for the treatment of mood states that are facilitated by suppressing neurotransmission. The compounds of Formula I may advantageously be used in conjunction with one or more other therapeutic agents, for instance, different antidepressant agents such as tricyclic antidepressants (e.g., amitriptyline, dothiepin, doxepin, trimipramine, butriptyline, clomipramine, desipramine, imipramine, iprindole, lofepramine, nortriptyline or protriptyline), monoamine oxidase inhibitors (e.g. isocarboxazid, phenelzine or tranylcyclopramine) or 5-HT re-uptake inhibitors (e.g., fluvoxamine, sertraline, fluoxetine or paroxetine), and/or with antiparkinsonian agents such as dopaminergic antiparkinsonian agents (e.g., levodopa, preferably in combination with a peripheral decarboxylase inhibitor e.g., benserazide or carbidopa, or with a dopamine agonist e.g., bromocriptine, lysuride or pergolide). It is to be understood that the present invention covers the use of a compound of Formula I or a physiologically acceptable salt thereof in combination with one or more other therapeutic agents.

Compounds of Formula I and pharmaceutically acceptable salts thereof, may also be combined with a 5-HT re-uptake inhibitor (e.g., fluvoxamine, sertraline, fluoxetine or paroxetine) or a pharmaceutically acceptable salt or polymorph thereof to treat disorders the treatment of which is facilitated by modulating serotonergic neurotransmission. Such treatment concerns diseases or disorders that include hypertension, depression, chemical dependencies, anxiety disorders (including panic disorder, generalized anxiety disorder, agoraphobia, simple phobias, and social phobia), post-traumatic stress disorder, obsessive-compulsive disorder, avoidant personality disorder and sexual dysfunction (including premature ejaculation), eating disorders, obesity, cluster headache, migraine, pain, Alzheimer's disease, obsessive-compulsive disorder, panic disorder, memory disorders (including dementia, amnestic disorders, and age-associated memory impairment), Parkinson's diseases (including dementia in Parkinson's disease, neuroleptic-induced Parkinsonism and tardive dyskinesias), endocrine disorders (including hyperprolactinaemia), vasospasm (particularly in the cerebral vasculature), cerebellar ataxia, gastrointestinal tract disorders (involving changes in motility and secretion) chronic paroxysmal hemicrania and headache (associated with vascular disorders).

The compounds of this invention can be administered via either the oral, parenteral (such as subcutaneous, intraveneous, intramuscular, intrasternal and infusion techniques), rectal, intranasal or topical routes to mammals. In general, these compounds are most desirably administered to humans in doses ranging from about 1 mg to about 2000 mg per day, although variations will necessarily occur depending upon the weight and condition of the subject being treated and the particular route of administration chosen. However, a dosage level that is in the range of from about 0.1 mg to about 20 mg per kg of body weight per day is most desirably employed. Nevertheless, variations may still occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effects provided that such higher dose levels are first divided into several small doses for administration throughout the day.

Pharmaceutically acceptable salts of the compounds of Formula I include the acid addition and base salts thereof.

Suitable acid addition salts are formed from acids which form non-toxic salts. Examples include the acetate, adipate, aspartate, benzoate, besylate, bicarbonate/carbonate, bisulfate/sulfate, borate, camphorsulfonate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mandelates mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, salicylate, saccharate, stearate, succinate, sulfonate, stannate, tartrate, tosylate, trifluoroacetate and xinafoate salts.

Suitable base salts are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002).

Pharmaceutically acceptable salts of compounds of Formula I may be prepared by one or more of three methods:
(i) by reacting the compound of Formula I with the desired acid or base;
(ii) by removing an acid- or base-labile protecting group from a suitable precursor of the compound of Formula I or by ring-opening a suitable cyclic precursor, for example, a lactone or lactam, using the desired acid or base; or
(iii) by converting one salt of the compound of Formula I to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionization in the resulting salt may vary from completely ionized to almost non-ionized.

The compounds of the present invention may be administered alone or in combination with pharmaceutically acceptable carriers or diluents by any of the above routes previously indicated, and such administration can be carried out in single or multiple doses. More particularly, the novel therapeutic agents of the invention can be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the therapeutically effective compounds of this invention are present in such dosage forms at concentration levels ranging about 5.0% to about 70% by weight.

For oral administration, tablets containing various excipients such as microcrystalline cellulose, sodium citrate, calcium carbonate, dicalcium phosphate and glycine may be employed along with various disintegrants such as starch and preferably corn, potato or tapioca starch, alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a compound of the present invention in either sesame or peanut oil or in aqueous propylene glycol may be employed. The aqueous solutions should be suitably buffered (preferably pH>8) if necessary and the liquid diluent first rendered isotonic. These aqueous solutions are suitable for intravenous injection purposes. The oily solutions are suitable for intra-articular, intra-muscular and subcutaneous injection purposes. The preparation of all these solutions under sterile conditions is readily accomplished by standard pharmaceutical techniques well-known to those skilled in the art.

### Biological Assay for Kv7.2/3 Channel Opening Activity

Plasmid Constructs:

Human Kv7.2 and Kv7.3 clones were obtained from GeneDynamics (Eugene, Oreg.). Both human Kv7.2/pIRESneo3 and human Kv7.3/pIREShygro3 expression vectors were constructed using a combination of fragments amplified from a human hippocampus library (extending from a NheI site near the 5′ end through the stop codon) and synthetic oligonucleotides (extending from the start codon to the BamHI site). The entire construct for Kv7.2 and Kv7.3 was subcloned into the pIRESneo3 and pIREShygro3 expression vectors (Clonetech, Mountain View, Calif.), respectively, using NheI/BamHI sites introduced on either side of the start and stop codons. The construct was sequenced in its entirety to make sure that no mutations were introduced during the amplification and cloning process. Adapted from Wickendon, A D et al., Mol Pharmacol 58(3):591-600, 2000.

Construction of CHO-K1 Cell Line Expressing Human Kv7.2 and Kv7.3 Voltage-Gated Potassium Channel Subunits

Transfection Vectors

Human Kv7.2/pIRESneo3 plasmid DNA (containing the Kv7.2 gene, accession # NM_{—}172107).

Human Kv7.3/pIREShyg3 plasmid DNA (containing the Kv7.3 gene, accession # NM_{—}004519).

Cell Line Construction:

Chinese hamster ovary (CHO-K1) cells were transfected with human-Kv7.2 in a pIRESneo3 plasmid DNA vector (Clonetech, Mountain View, Calif.) and the h-Kv7.3 subunit in pIREShygro3 plasmid DNA vector using Lipofectamine-2000™ reagent (InVitrogen, San Diego, Calif.), according to the manufacturers instructions. Cells stably expressing the human Kv7.2 and Kv7.3 constructs were identified by their resistance to 400 ug/ml, geneticin (Gibco #10131-027) and 400 ug/ml, hygromycin-B (Invitrogen #10687-010). Clones were screened for functional expression using the whole-cell, voltage-clamp technique.

Biological Assay

To determine if compounds can enhance voltage-dependent K-current in Kv7.2/7.3 channel containing CHO-K1 cells. Planar patch-clamp is used on IonWorks to functionally determine percent enhancement of Kv7.2/7.3-current at 0 mV (compared to retigabine) and potency (EC₅₀) of compounds. Intrinsic activity and/or potency may be important in determining in vivo pharmacological efficacy of the compounds.

Compound Preparation:

Serial dilutions were made in DMSO on an Apricot Personal Pipettor. Compounds were then diluted in external buffer on a 384 well assay plate (final DMSO concentration=0.3%).

Method:

Cells used in this assay were CHO-K1 expressing Kv7.2/7.3 channels. Cells were maintained in growth media containing: F-12 (Gibco #11765-054), 10% FBS (Invitrogen 16140-071), 1:100 Glutamax (Gibco #35050-061), 1:100 Penicillin/streptomycin (Gibco #15140-122), 400 ug/ml Geneticin (Gibco #10131-027), 400 ug/ml Hygromycin-B (Invitrogen #10687-010). Cells were grown in T-150 flasks to a confluence of approx. 80%. External recording buffer contained (in mM): NaCl (137), KCl (4), MgCl₂ (1), CaCl₂ (1.8), HEPES (10), and glucose (10), pH was adjusted to 7.3 with NaOH and osmolarity was adjusted to 300-305 mOsM with sucrose, if necessary. Internal buffer contained (in mM): Kgluconate (120), KCl (20), NaCl (5), MgCl₂ (1), CaCl₂ (2), HEPES (10), KF (2), and Na2ATP (2). Na2ATP was added to internal buffer right before use and pH was adjusted to 7.2 with KOH. Osmolarity of internal buffer was adjusted to 290-295 mOsM.

Cells were washed 1× with Ca/Mg free PBS and then removed from the plates with a 50:50 mixture of versene (Gibco 15040): 0.25% trypsin-EDTA (Gibco 25200) (4 min), triturated, centrifuged at ˜1000 rpm for 5 min, and resuspended at ˜2.5 million cells/ml in external buffer for recording on IonWorks. Potassium current measurements were made using an IonWorks Quattro instrument (MDS Corp.) using PatchPlate PPC substrates with 64 apertures per well. IonWorks calculated leak current was digitally subtracted from the total current acquired. Potassium current was elicited by stepping from −80 mV to 0 mV (2 sec) was measured in the absence and presence of increasing concentrations (½ log) of unknown compound (7-point concentration curves). Retigabine was run as a positive control and comparator on each PatchPlate PPC. Maximum increase in K current was determined by subtracting the current elicited in external buffer alone wells from the current elicited in wells with compound treatment (both at 0 mV). A sample size of 8 wells per treatment condition was used. Six compounds could be run on each PatchPlate. Compound dilutions were made in 384 well assay plates using an Apricot Personal Pipettor (Apricot Designs, Inc.). Pre-scan vs post-scan current rundown (˜5-20%) in control wells was calculated and subtracted from the compound treated wells. Wells with pre-scan seal resistances of <40 Mohm or currents of <50 pA were excluded from the analysis.

Maximum potassium current enhancement, as a % of max retigabine enhancement, was reported as well as EC₅₀ values for each compound. EC₅₀ values in nM units. Compounds of the invention analyzed by this assay have been found to have significant activity in opening Kv7.2/3 channels with EC₅₀ values <100 uM.

| | | Kv7.2/3 |
|---|---|---|
| Example | Compound Name | EC₅₀ |
| 1 | N-{2-[(2R)-Butan-2-ylamino]-9-methyl-4-oxo- | 168 nM |
| | 4H-pyrido[1,2-a]pyrimidin-3-yl}-3,3- | |
| | dimethylbutanamide | |
| 2a | N-{2-[(1-Cyclopropylethyl)amino]-9-methyl-4- | 227 nM |
| | oxo-4H-pyrido[1,2-a]pyrimidin-3-yl}-3,3- | |
| | dimethylbutanamide, (+)-enantiomer | |
| 2b | N-{2-[(1-Cyclopropylethyl)amino]-9-methyl-4- | 128 nM |
| | oxo-4H-pyrido[1,2-a]pyrimidin-3-yl}-3,3- | |
| | dimethylbutanamide, (−)-enantiomer | |
| 3 | 3,3-Dimethyl-N-[9-methyl-4-oxo-2-(propan-2- | 450 nM |
| | ylamino)-4H-pyrido[1,2-a]pyrimidin-3- | |
| | yl]butanamide | |
| 4 | N-[2-Cyclopropyl-4-oxo-9-(trifluoromethyl)-4H- | 137 nM |
| | pyrido[1,2-a]pyrimidin-3-yl]-3,3- | |
| | dimethylbutanamide | |
| 5a | N-[2-Cyclopropyl-4-oxo-9-(trifluoromethyl)- | 1240 nM |
| | 6,7,8,9-tetrahydro-4H-pyrido[1,2-a]pyrimidin-3- | |
| | yl]-3,3-dimethylbutanamide, (−)-enantiomer | |
| 5b | N-[2-Cyclopropyl-4-oxo-9-(trifluoromethyl)- | 298 nM |
| | 6,7,8,9-tetrahydro-4H-pyrido[1,2-a]pyrimidin-3- | |
| | yl]-3,3-dimethylbutanamide, (+)-enantiomer | |
| 6 | N-[2-(Cyclobutylamino)-4-oxo-9- | 91.5 nM |
| | (trifluoromethyl)-4H-pyrido[1,2-a]pyrimidin-3- | |
| | yl]-3,3-dimethylbutanamide | |
| 7 | 3,3-Dimethyl-N-[4-oxo-2-(propan-2-ylamino)-9- | 119 nM |
| | (trifluoromethyl)-4H-pyrido[1,2-a]pyrimidin-3- | |
| | yl]butanamide | |
| 8a | 3,3-Dimethyl-N-[4-oxo-2-(propan-2-ylamino)-9- | 382 nM |
| | (trifluoromethyl)-6,7,8,9-tetrahydro-4H- | |
| | pyrido[1,2-a]pyrimidin-3-yl]butanamide, (−) | |
| | enantiomer | |
| 8b | 3,3-Dimethyl-N-[4-oxo-2-(propan-2-ylamino)-9- | 412 nM |
| | (trifluoromethyl)-6,7,8,9-tetrahydro-4H- | |
| | pyrido[1,2-a]pyrimidin-3-yl]butanamide, (+) | |
| | enantiomer | |
| 9 | N-[9-(1,1-Difluoroethoxy)-4-oxo-2-(propan-2- | 369 nM |
| | ylamino)-4H-pyrido[1,2-a]pyrimidin-3-yl]-3,3- | |
| | dimethylbutanamide | |
| 10 | N-[2-Cyclopropyl-9-(1,1-difluoroethoxy)-4-oxo- | 306 nM |
| | 4H-pyrido[1,2-a]pyrimidin-3-yl]-3,3- | |
| | dimethylbutanamide | |
| 11a | N-{2-[(2R)-Butan-2-ylamino]-9-methyl-4-oxo- | 307 nM |
| | 6,7,8,9-tetrahydro-4H-pyrido[1,2-a]pyrimidin-3- | |
| | yl}-3,3-dimethylbutanamide, diastereomer 1 | |
| 11b | N-{2-[(2R)-Butan-2-ylamino]-9-methyl-4-oxo- | 942 nM |
| | 6,7,8,9-tetrahydro-4H-pyrido[1,2-a]pyrimidin-3- | |
| | yl}-3,3-dimethylbutanamide, diastereomer 2 | |

### General Synthetic Schemes and Working Examples

The compounds of Formula I may be prepared by the methods described below, together with synthetic methods known in the art of organic chemistry, or modifications and derivatizations that are familiar to those of ordinary skill in the art. The starting materials used herein are commercially available or may be prepared by routine methods known in the art (such as those methods disclosed in standard reference books such as the Compendium of Organic Synthetic Methods, Vol. I-XII (published by Wiley-Interscience)). Preferred methods include, but are not limited to, those described below. The following schemes and examples are exemplary of the processes for making compounds of Formula I. It is to be understood, however, that the invention, as fully described herein and as recited in the claims, is not intended to be limited by the details of the following examples.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991; and T. W. Greene and P. G. M. Wuts, Protective Groups in Organic Chemistry, John Wiley & Sons, 1999, which are hereby incorporated by reference.

Compounds of Formula I, or their pharmaceutically acceptable salts, can be prepared according to the reaction Schemes discussed herein below. Unless otherwise indicated, the substituents in the Schemes are defined as above. Isolation and purification of the products is accomplished by standard procedures, which are known to a chemist of ordinary skill.

It will be understood by one skilled in the art that the various symbols, superscripts and subscripts used in the schemes, methods and examples are used for convenience of representation and/or to reflect the order in which they are introduced in the schemes, and are not intended to necessarily correspond to the symbols, superscripts or subscripts in the appended claims. The schemes are representative of methods useful in synthesizing the compounds of the present invention. They are not to constrain the scope of the invention in any way.

The compounds of Formula Ia, wherein R² is cycloalkyl and —X—Y— is ═CH—CH═, can be prepared by several methods known to those skilled in the art. One such method is depicted in Scheme 1 starting with 2-substituted aminopyridines of Formula III, which may be obtained from commercial sources or by methods known to those skilled in the art. Hydroxy pyrimidones of Formula V may be obtained through cyclization with a suitable malonate derivative (IV), for instance by reaction with neat dialkyl malonates at elevated temperature. Alternatively, cyclization may be carried out with activated malonates, for example bis(2,4,6-trichlorophenyl) malonate (Org. Biomol. Chem. 2009, 7, 3940-3946), in a suitable solvent such as but not limited to tetrahydrofuran (THF), at a reaction temperature ranging from RT to reflux. The hydroxypyrimidones of Formula V may be converted to the corresponding chloropyrimidones of Formula VI using a chlorination reagent, such as but not limited to phosphorous oxychloride (POCl₃) or sulfonyl chloride (SOCl₂). Suzuki coupling of chloropyrimidones of Formula VI with cycloalkylboronic acids, R²B(OH)₂, by several methods known to those skilled in the art, specifically in the presence of palladium(II) acetate, 2-dicyclohexylphosphino-2′,6′-dimethoxybiphenyl (S-Phos), and a suitable base such as but not limited to potassium phosphate in an inert solvent such as toluene in the presence or absence of from about 1% to about 10% water, preferably about 5% water, can then yield compounds of Formula VII. The compounds of Formula VII may be nitrated by several methods known to those skilled in the art, for instance using a mixture of concentrated sulfuric acid and fuming nitric acid at a temperature ranging from −78° C. to RT, to yield nitropyrimidones of Formula VIII, which may be reduced to the corresponding aminopyrimidones of Formula IX using a variety of reduction methods known to one skilled in the art, such as treatment with iron powder and calcium chloride in aqueous ethanol at reflux temperature. The resulting primary amines of Formula IX may be acylated to yield compounds of Formula Ia using a variety of methods known to one skilled in the art, for instance using an acid chloride in the presence of a base such as potassium phosphate in a solvent such as THF, with or without 4-(dimethylamino)pyridine at temperatures ranging from ambient to 80° C. Alternatively compounds of Formula Ia may also be accessed via amide coupling conditions known to one skilled in the art, by treatment of compounds of Formula IX with carboxylic acids and amide coupling reagents.

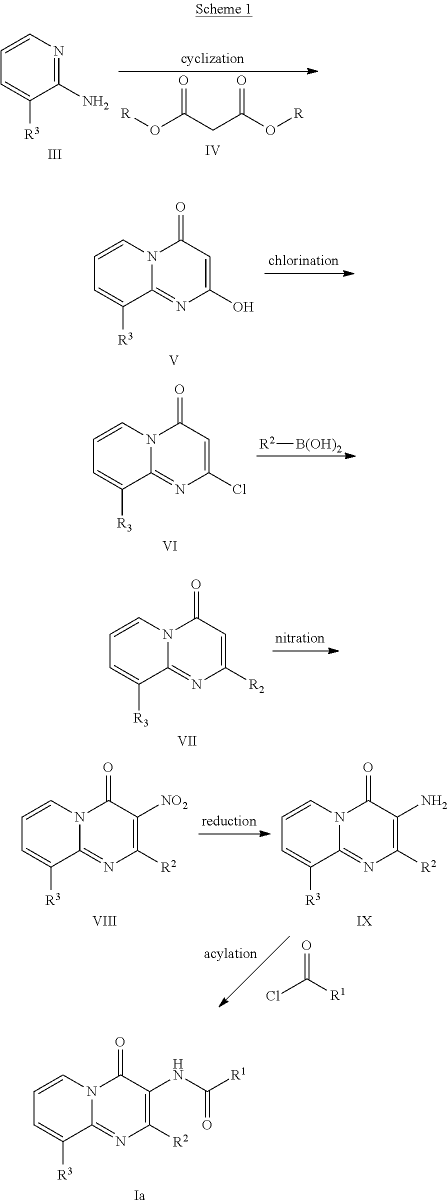

The compounds of Formula Ib, wherein R² is NR⁴R⁵ and —X—Y— is ═CH—CH═, can be prepared by the method shown in Scheme 2 starting with chloropyrimidones of Formula VI, which may be obtained from the method described in Scheme 1. The compounds of Formula VI may be nitrated by several methods known to those skilled in the art, for example with nitronium tetrafluoroborate in a solvent such as sulfolane and at temperatures ranging from −78° C. to RT, to yield nitropyrimidones of Formula X, which may be reduced to the corresponding aminopyrimidones of Formula XI using a variety of reduction methods known to one skilled in the art, such as treatment with iron powder and calcium chloride in aqueous ethanol at reflux temperature. The resulting primary amines of Formula XI may be acylated to yield compounds of Formula XII using a variety of methods known to one skilled in the art, for instance using an acid chloride in the presence of a base such as potassium phosphate in a solvent such as THF at temperatures ranging from ambient to 80° C. The compounds of Formula Ib may be prepared by treating chloropyrimidones of formula XII with the corresponding amines, NHR⁴R⁵, in the presence of a suitable base such as but not limited to triethylamine in a suitable solvent such as but not limited to EtOAc at an elevated temperature with or without microwave heating.

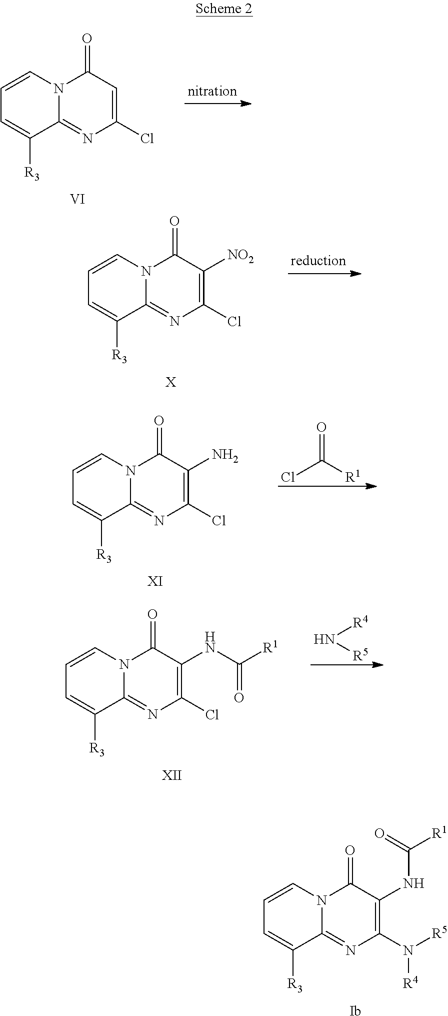

The preparations of the compounds of Formula Ic and Id are shown in Scheme 3. The compounds of Formula Ic, wherein —X—Y— is —CH₂—CH₂—, can be prepared via method A by hydrogenation of the compounds of Formula Ia and Ib in the presence of a suitable catalyst, such as but not limited to 10% Palladium on carbon in a suitable solvent such as but not limited to methanol under a hydrogen atmosphere of 20 to 60 psi. The compounds of Formula Id, wherein R² is NR⁴R⁵ and —X—Y— is —CH₂—CH₂—, or —CH₂— may be prepared through method B starting from cyclic amidines of Formula XIII, which may be obtained from commercial sources or by methods known to those skilled in the art. Hydroxypyrimidones of Formula XIV may be obtained through cyclization with malonate derivatives of Formula XVI, which may be commercially available or obtained by methods known to those skilled in the art. The hydroxypyrimidones of Formula XIV may be converted to the corresponding chloropyrimidones of Formula XV using a chlorination reagent, such as but not limited to phosphorous oxychloride (POCl₃) or sulfonyl chloride (SOCl₂). The compounds of Formula Id may be prepared by treating compounds XV with the corresponding amines, NHR⁴R⁵, in the presence of a suitable base such as but not limited to triethylamine in a suitable solvent such as but not limited to EtOAc or 3-methyl-1-butanol at an elevated temperature with or without microwave irradiation.

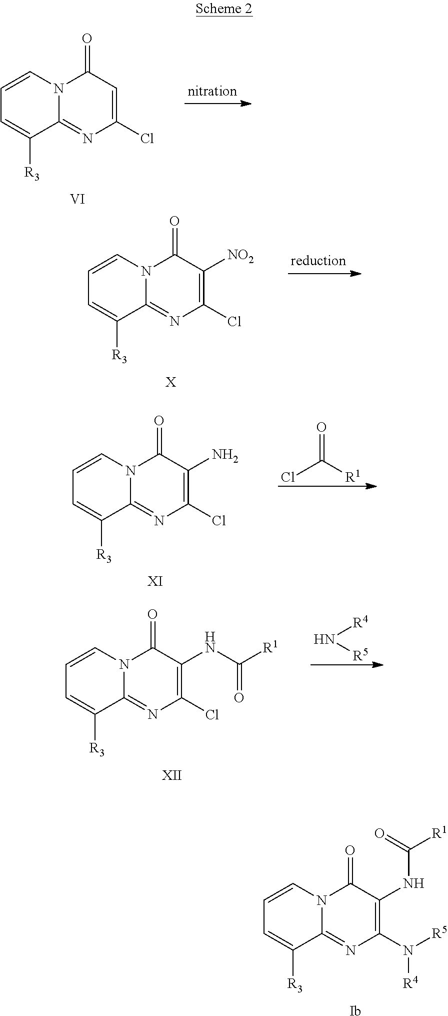

### EXAMPLES AND EXPERIMENTAL PROCEDURES

The following illustrate the synthesis of compounds of the present invention. Additional compounds within the scope of this invention may be prepared using the methods illustrated in these Examples, either alone or in combination with techniques generally known in the art.

Experiments were generally carried out under inert atmosphere (nitrogen or argon), particularly in cases where oxygen- or moisture-sensitive reagents or intermediates were employed. Commercial solvents and reagents were generally used without further purification unless indicated otherwise, including anhydrous solvents where appropriate (generally Sure-Seal™ products from the Aldrich Chemical Company, Milwaukee, Wis.). Mass spectrometry data is reported from liquid chromatography-mass spectrometry (LCMS). Chemical shifts for nuclear magnetic resonance (NMR) data are expressed in parts per million (ppm, 6) referenced to residual peaks from the deuterated solvents employed.

For syntheses of other compounds of Formula I not specifically exemplified, reaction conditions (length of reaction and temperature) may vary. In general, reactions were followed by thin layer chromatography or mass spectrometry, and subjected to work-up when appropriate such that reaction times are approximate. Purifications may vary between experiments and in general, solvents and the solvent ratios used for eluants/gradients were chosen to provide appropriate retention times.

### Example 1

### N-{2-[(2R)-Butan-2-ylamino]-9-methyl-4-oxo-4H-pyrido[1,2-a]pyrimidin-3-yl}-3,3-dimethylbutanamide

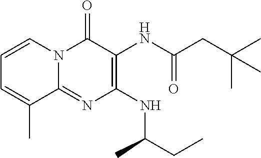 [SMILES: CC[C@@H](C)Nc1nc2c(C)cccn2c(=O)c1NC(=O)CC(C)(C)C]

### Step 1. 2-Hydroxy-9-methyl-4H-pyrido[1,2-a]pyrimidin-4-one

A mixture of 2-amino-3-picoline (10.8 g, 100 mmol) and diethyl malonate (76.3 mL, 500 mmol) was stirred at 150° C. for 24 h. The mixture was cooled to RT, diluted with ethyl acetate (150 mL), and filtered. The solid was washed with ethyl acetate (100 mL) to give the titled compound (17.0 g, 96.5%) as a white solid.

¹H NMR (DMSO-d₆) δ (ppm) 11.43 (br s, 1H), 8.76 (d, J=7.0 Hz, 1H), 7.79 (d, J=6.6 Hz, 1H), 7.15-7.10 (m, 1H), 5.36 (br, 1H), 2.40 (s, 3H)

MS (ES+) 177

### Step 2. 2-Chloro-9-methyl-4H-pyrido[1,2-a]pyrimidin-4-one

A mixture of 2-hydroxy-9-methyl-4H-pyrido[1,2-a]pyrimidin-4-one (17.0 g, 96 mmol) and POCl₃ (45 mL, 482 mmol) was stirred at 100° C. for 3 h. The mixture was dropped into stirring water at RT (intermittently cooled with an ice-water bath to maintain the temperature). The mixture was neutralized with 20% aq. NaOH. The suspension was filtered, and the solid was washed with water to give the titled compound (14.5 g, 77%) as a pale brown solid.

¹H NMR (CDCl₃) δ (ppm) 8.92 (d, J=7.2 Hz, 1H), 7.67 (d, J=6.8 Hz, 1H), 7.14-7.08 (m, 1H), 6.44 (s, 1H), 2.57 (s, 3H)

MS (ES+) 195, 197

### Step 3. 2-Chloro-9-methyl-3-nitro-4H-pyrido[1,2-a]pyrimidin-4-one

A mixture of 2-chloro-9-methyl-4H-pyrido[1,2-a]pyrimidin-4-one (14.5 g, 74.5 mmol) and 0.5 M nitronium tetrafluoroborate in sulfolane (298 mL, 149 mmol) was stirred at RT for 2 h. The mixture was added drop-wise into ice-cooled water (30 mL) and then neutralized with 1 N aq. NaOH. The suspension was filtered and the solid was washed with water to give the titled compound (12.5 g, 70%) as a pale yellow solid.

¹H NMR (CDCl₃) δ (ppm) 9.03 (d, J=6.8 Hz, 1H), 7.90 (d, J=7.0 Hz, 1H), 7.36-7.31 (m, 1H), 2.65 (s, 3H)

MS (ES+) 240, 242

### Step 4. 3-Amino-2-chloro-9-methyl-4H-pyrido[1,2-a]pyrimidin-4-one

To a mixture of 2-chloro-9-methyl-3-nitro-4H-pyrido[1,2-a]pyrimidin-4-one (12.5 g, 52.2 mmol), iron powder (8.74 g, 156 mmol), and CaCl₂ (11.6 g, 104 mmol) in ethanol (261 mL) at RT was added water (52.2 mL). The mixture was refluxed for 1 h and then filtered through a pad of Celite with dichloromethane/methanol (10:1). The filtrate was washed with 1 N aq. NaOH, and the organic layer was dried over MgSO₄ and filtered. The filtrate was concentrated in vacuo to give the titled compound (9.3 g, 85%) as a yellow solid.

¹H NMR (CDCl₃) δ (ppm) 8.71 (d, J=7.2 Hz, 1H), 7.30 (d, J=6.6 Hz, 1H), 6.96-6.90 (m, 1H), 2.52 (s, 3H)

MS (ES+) 210, 212

### Step 5. N-(2-Chloro-9-methyl-4-oxo-4H-pyrido[1,2-a]pyrimidin-3-yl)-3,3-dimethylbutanamide

tert-Butylacetyl chloride (6.2 mL, 44 mmol) was added drop-wise to a mixture of 3-amino-2-chloro-9-methyl-4H-pyrido[1,2-a]pyrimidin-4-one (9.3 g, 44 mmol) and K₃PO₄ (28.2 g, 133 mmol) in THF (88 mL) at RT. The mixture was stirred at 50° C. for 16 h. The mixture was quenched with 1 N aq. HCl (160 mL) and extracted three times with dichloromethane/methanol (10:1). The combined extracts were dried over MgSO₄ and filtered. The filtrate was concentrated in vacuo, and the residue was triturated with cold ethyl acetate (200 mL) to give the titled compound (11.1 g, 81%) as a pale yellow solid.

¹H NMR (CDCl₃) δ (ppm) 8.85 (d, J=7.2 Hz, 1H), 7.64 (d, J=6.8 Hz, 1H), 7.14-7.07 (m, 1H), 6.88 (br s, 1H), 2.57 (s, 3H), 2.30 (s, 2H), 1.14 (s, 9H)

MS (ES+) 308, 310

### Step 6

### Example 1

To a mixture of N-(2-chloro-9-methyl-4-oxo-4H-pyrido[1,2-a]pyrimidin-3-yl)-3,3-dimethylbutanamide (8.0 g, 26 mmol) and triethylamine (5.4 mL, 39 mmol) in ethyl acetate (52 mL) in a sealed tube was added (R)-2-aminobutane (2.85 g, 39 mmol). The mixture was stirred at 60° C. for 16 h and 150° C. for 30 min. The mixture was treated with sat. aq. NaHCO₃ and extracted twice with dichloromethane. The combined extracts were dried over MgSO₄ and filtered, and the filtrate was concentrated in vacuo. The crude material was purified by silica gel column chromatography (0-80% ethyl acetate in heptane, gradient). The product was washed with cold tert-butyl methyl ether/ethanol (3:1) to give Example 1 (5.9 g, 66%) as a white solid.

¹H NMR (CDCl₃) δ (ppm) 8.67 (d, J=7.0 Hz, 1H), 7.51 (br s, 1H), 7.38 (d, J=6.6 Hz, 1H), 6.81-6.74 (m, 1H), 6.26 (br s, 1H), 4.28-4.18 (m, 1H), 2.43 (s, 3H), 2.33 (s, 2H), 1.69-1.54 (m, 2H), 1.25 (d, J=6.4 Hz, 3H), 1.11 (s, 9H), 0.95 (t, J=7.4 Hz, 3H)

MS (ES+) 345

[α]_{D}²⁰=−28° (c 0.17, methanol)

### Example 2

### N-{2-[(1-Cyclopropylethyl)amino]-9-methyl-4-oxo-4H-pyrido[1,2-a]pyrimidin-3-yl}-3,3-dimethylbutanamide

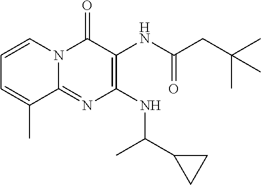 [SMILES: Cc1cccn2c(=O)c(NC(=O)CC(C)(C)C)c(NC(C)C3CC3)nc12]

A mixture of N-(2-chloro-9-methyl-4-oxo-4H-pyrido[1,2-a]pyrimidin-3-yl)-3,3-dimethylbutanamide (0.60 g, 1.9 mmol, Example 1, Step 5), diisopropylethylamine (1.5 mL, 8.6 mmol), and (1-cyclopropylethyl)amine (0.54 g, 4.4 mmol) in ethanol (2.0 mL) was heated with microwave irradiation (150° C., 60 min). The mixture was diluted with ethyl acetate and washed with sat. aq. NaHCO₃. The organic layer was dried over MgSO₄ and filtered. The filtrate was concentrated in vacuo. The crude material was purified by silica gel column chromatography (0-80% EtOAc in heptane, gradient) to give the desired racemic product (0.60 g, 87%).

¹H NMR (CDCl₃) δ (ppm) 8.68 (d, J=7.0 Hz, 1H), 7.46 (br s, 1H), 7.37 (d, J=6.6 Hz, 1H), 6.80-6.73 (m, 1H), 6.28 (br s, 1H), 3.84-3.72 (m, 1H), 2.41 (s, 3H), 2.32 (s, 2H), 1.32 (d, J=6.6 Hz, 3H), 1.12 (s, 9H), 1.03-0.91 (m, 1H), 0.53-0.38 (m, 3H), 0.30-0.22 (m, 1H)

MS (ES+) 357

The individual enantiomers were isolated by supercritical fluid chromatography (Chiralcel OD-H, mobile phase 85:15 CO₂1 methanol).

### Example 2a

Earlier-eluting peak: [α]_{D}²⁰=+7.2° (c 0.93, methanol)

### Example 2b

Later-eluting peak: [α]_{D}²⁰=−12.7° (c 0.83, methanol)

### Example 3

### 3,3-Dimethyl-N-[9-methyl-4-oxo-2-(propan-2-ylamino)-4H-pyrido[1,2-a]pyrimidin-3-yl]butanamide

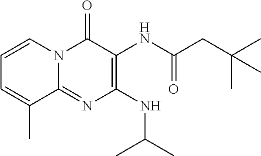 [SMILES: Cc1cccn2c(=O)c(NC(=O)CC(C)(C)C)c(NC(C)C)nc12]

A mixture of N-(2-chloro-9-methyl-4-oxo-4H-pyrido[1,2-a]pyrimidin-3-yl)-3,3-dimethylbutanamide (2.0 g, 6.5 mmol) and isopropylamine (4.8 mL, 56 mmol) in ethyl acetate (10 mL) was stirred at 60° C. for 19 h. The mixture was treated with sat. aq. NaHCO₃ and extracted with ethyl acetate. The extract was dried over MgSO₄ and filtered, and the filtrate was concentrated in vacuo. The crude material was purified by crystallization from ethanol/diethyl ether (1:2) to give Example 3 (1.7 g, 80%) as a yellow solid.

¹H NMR (CDCl₃) δ (ppm) 8.69 (d, J=7.0 Hz, 1H), 7.46 (br s, 1H), 7.39 (d, J=6.6 Hz, 1H), 6.82-6.75 (m, 1H), 6.23 (br s, 1H), 4.44-4.31 (m, 1H), 2.45 (s, 3H), 2.33 (s, 2H), 1.29 (d, J=6.4 Hz, 6H), 1.11 (s, 9H)

MS (ES+) 331

### Example 4

### N-[2-Cyclopropyl-4-oxo-9-(trifluoromethyl)-4H-pyrido[1,2-a]pyrimidin-3-yl]-3,3-dimethylbutanamide

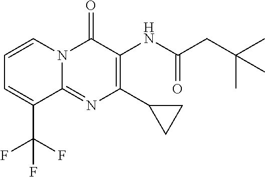 [SMILES: CC(C)(C)CC(=O)Nc1c(C2CC2)nc2c(C(F)(F)F)cccn2c1=O]

### Step 1. 2-Hydroxy-9-(trifluoromethyl)-4H-pyrido[1,2-a]pyrimidin-4-one

To a stirring solution of 3-(trifluoromethyl)pyridin-2-amine (50.0 g, 308 mmol) in THF (616 mL) at RT was added bis(2,4,6-trichlorophenyl)malonate (143 g, 308 mmol, Org. Biomol. Chem. 2009, 7, 3940-3946). The mixture was refluxed for 16 h, then was concentrated to a slurry. The slurry was triturated with ethyl acetate (200 mL), filtered, and washed with ethyl acetate to give the titled compound (55.3 g, 77.8%) as a white solid.

¹H NMR (DMSO-d₆) δ (ppm) 9.13 (d, J=7.0 Hz, 1H), 8.29 (d, J=7.0 Hz, 1H), 7.25 (t, J=7.0 Hz, 1H), 5.69 (s, 1H) (OH was not observed)

MS (ES+) 231

### Step 2. 2-Chloro-9-(trifluoromethyl)-4H-pyrido[1,2-a]pyrimidin-4-one

Following the procedure of Example 1, Step 2, 2-hydroxy-9-(trifluoromethyl)-4H-pyrido[1,2-a]pyrimidin-4-one (28 g, 122 mmol) was reacted with POCl₃ (90 mL, 970 mmol) to afford the titled compound (20.9 g, 73%).

¹H NMR (CDCl₃) δ (ppm) 9.17 (d, J=6.8 Hz, 1H), 8.20 (d, J=7.0 Hz, 1H), 7.29-7.21 (m, 1H), 6.57 (s, 1H)

MS (ES+) 249, 251

### Step 3. 2-Cyclopropyl-9-(trifluoromethyl)-4H-pyrido[1,2-a]pyrimidin-4-one

A mixture of 2-chloro-9-(trifluoromethyl)-4H-pyrido[1,2-a]pyrimidin-4-one (20.9 g, 84.1 mmol), cyclopropylboronic acid (9.39 g, 109 mmol), S-Phos (3.45 g, 8.41 mmol), and K₃PO₄ (62.5 g, 294 mmol) in toluene (336 mL), water (16.8 mL) and Pd(OAc)₂ (944 mg, 4.2 mmol) were added. The mixture was degassed and filled with nitrogen. The mixture was stirred at 90° C. for 2 h, at which time it was cooled, diluted with dichloromethane (500 mL) and filtered through a pad of Celite. The filtrate was concentrated in vacuo. The crude material was purified by silica gel column chromatography (0-10% ethyl acetate in dichloromethane, gradient) to give the titled compound (19.6 g, 91.7%) as a brown solid.

¹H NMR (CDCl₃) δ (ppm) 9.08 (d, J=7.0 Hz, 1H), 7.99 (d, J=7.0 Hz, 1H), 7.01 (dd, J=7.0 Hz, 1H), 6.46 (s, 1H), 1.96-1.87 (m, 1H), 1.28-1.20 (m, 2H), 1.06-0.98 (m, 2H)

MS (ES+) 255

### Step 4. 2-Cyclopropyl-3-nitro-9-(trifluoromethyl)-4H-pyrido[1,2-a]pyrimidin-4-one

To a mixture of 2-cyclopropyl-9-(trifluoromethyl)-4H-pyrido[1,2-a]pyrimidin-4-one (19.6 g, 77.1 mmol) and conc. H₂SO₄ (86.5 mL) at 0° C. was added fuming HNO₃ (14.5 mL). The mixture was stirred at RT for 2 h. The mixture was added drop-wise to stirring water (600 mL) at 0° C. and stirred for 30 min. The suspension was filtered, and the collected solid was washed with water to give the titled compound (quant.) as a yellow solid.

¹H NMR (CDCl₃) δ (ppm) 9.15 (d, J=7.0 Hz, 1H), 8.19 (d, J=7.0 Hz, 1H), 7.27-7.20 (m, 1H), 2.31-2.23 (m, 1H), 1.49-1.42 (m, 2H), 1.29-1.20 (m, 2H)

MS (ES+) 300

### Step 5. 3-Amino-2-cyclopropyl-9-(trifluoromethyl)-4H-pyrido[1,2-a]pyrimidin-4-one

Following the procedure of Example 1, Step 4, 2-cyclopropyl-3-nitro-9-(trifluoromethyl)-4H-pyrido[1,2-a]pyrimidin-4-one (23 g, 77 mmol) was converted to the titled compound (19.5 g, 94%).

¹H NMR (DMSO-d₆) δ (ppm) 8.81 (d, J=7.2 Hz, 1H), 8.86 (d, J=6.8 Hz, 1H), 7.10-7.03 (m, 1H), 5.28 (s, 2H), 2.33-2.24 (m, 1H), 1.04-0.92 (m, 4H)

MS (ES+) 270

### Step 6

### Example 4

Following the procedure of Example 1, Step 5, 3-amino-2-cyclopropyl-9-(trifluoromethyl)-4H-pyrido[1,2-a]pyrimidin-4-one (15.0 g, 55.7 mmol) was converted to Example 4 (10.3 g, 50.3%).

¹H NMR (CDCl₃) δ (ppm) 8.97 (d, J=7.0 Hz, 1H), 7.95 (d, J=7.6 Hz, 1H), 7.06-6.96 (m, 2H), 2.34 (s, 2H), 2.14-2.05 (m, 1H), 1.36-1.28 (m, 2H), 1.15 (s, 9H), 1.12-1.05 (m, 2H)

MS (ES+) 368 (ES−) 366

### Example 5

### N-[2-Cyclopropyl-4-oxo-9-(trifluoromethyl)-6,7,8,9-tetrahydro-4H-pyrido[1,2-a]pyrimidin-3-yl]-3,3-dimethylbutanamide

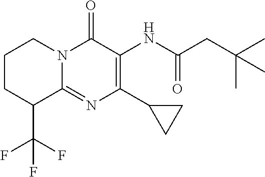 [SMILES: CC(C)(C)CC(=O)Nc1c(C2CC2)nc2n(c1=O)CCCC2C(F)(F)F]

A mixture of Example 4 (1.0 g, 33.5 mmol) and 10% Pd—C (135 mg) in methanol (50 mL) was hydrogenated at 50 psi for 6 h. The mixture was filtered through a pad of Celite, and the filtrate was concentrated in vacuo. The crude material was purified by silica gel column chromatography (20-100% EtOAc in heptane, gradient) to give the racemic product (0.65 g, 64%).

¹H NMR (CDCl₃) δ (ppm) 6.97 (br s, 1H), 4.04-3.81 (m, 2H), 3.59-3.45 (m, 1H), 2.29 (s, 2H), 2.22-2.00 (m, 3H), 1.94-1.79 (m, 2H), 1.18-0.90 (m, 13H)

MS (ES+) 372 (ES−) 370

The individual enantiomers were isolated by supercritical fluid chromatography (Chiralpak AD-H, mobile phase 80/20 CO₂/ethanol).

### Example 5a

Earlier-eluting peak: [α]_{D}²⁰=−23.7° (c 0.46, methanol)

### Example 5b

Later-eluting peak: [α]_{D}²⁰=+22.6° (c 1.75, methanol)

### Example 6

### N-[2-(Cyclobutylamino)-4-oxo-9-(trifluoromethyl)-4H-pyrido[1,2-a]pyrimidin-3-yl]-3,3-dimethylbutanamide

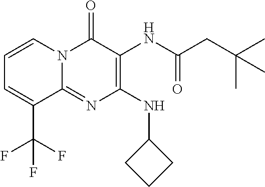 [SMILES: CC(C)(C)CC(=O)Nc1c(NC2CCC2)nc2c(C(F)(F)F)cccn2c1=O]

### Step 1. 2-Chloro-3-nitro-9-(trifluoromethyl)-4H-pyrido[1,2-a]pyrimidin-4-one

Following the procedure of Example 4, Step 4, 2-chloro-9-(trifluoromethyl)-4H-pyrido[1,2-a]pyrimidin-4-one (16.7 g, 67.2 mmol, Example 4, Step 2) was converted to the title compound (17.1 g, 86.7%).

¹H NMR (CDCl₃) δ (ppm) 9.27 (dd, J=7.1, 1.5 Hz, 1H), 8.40-8.44 (m, 1H), 7.48 (dd, J=7.0, 7.0 Hz, 1H)

### Step 2. N-[2-Chloro-4-oxo-9-(trifluoromethyl)-4H-pyrido[1,2-a]pyrimidin-3-yl]-3,3-dimethylbutanamide

Following the procedure of Example 1, Step 4, 2-chloro-3-nitro-9-(trifluoromethyl)-4H-pyrido[1,2-a]pyrimidin-4-one (16.7 g, 56.9 mmol) was converted to the crude primary amine. This in turn was used, via the procedure of Example 1, Step 5, to synthesize the titled compound (8.0 g, 39%).

¹H NMR (CDCl₃) δ (ppm) 9.09 (d, J=6.4 Hz, 1H), 8.15 (d, J=6.8 Hz, 1H), 7.27-7.19 (m, 1H), 6.85 (br s, 1H), 2.32 (s, 2H), 1.15 (s, 9H)

MS (ES+) 362, 364 (ES−) 360, 362

### Step 3

### Example 6

A mixture of N-[2-chloro-4-oxo-9-(trifluoromethyl)-4H-pyrido[1,2-a]pyrimidin-3-yl]-3,3-dimethylbutanamide (0.50 g, 1.4 mmol) and cyclobutylamine (0.30 g, 4.2 mmol) in THF (2.8 mL) was stirred at RT for 2 h. The mixture was diluted with ethyl acetate (50 mL) and washed with sat. aq. NaHCO₃ (30 mL). The organic layer was dried over MgSO₄ and filtered; the filtrate was concentrated in vacuo. The crude material was purified by silica gel column chromatography (10-75% ethyl acetate in heptane, gradient) to give Example 6 (0.47 g, 87%) as a pale yellow solid.

¹H NMR (CDCl₃) δ (ppm) 8.93 (d, J=7.0 Hz, 1H), 7.92 (d, J=6.8 Hz, 1H), 7.36 (br, 1H), 6.93-6.87 (m, 1H), 6.74 (br s, 1H), 4.62-4.50 (m, 1H), 2.47-2.37 (m, 2H), 2.34 (s, 2H), 2.05-1.92 (m, 2H), 1.83-1.73 (m, 2H), 1.13 (s, 9H)

MS (ES+) 397 (ES−) 395

### Example 7

### 3,3-Dimethyl-N-[4-oxo-2-(propan-2-ylamino)-9-(trifluoromethyl)-4H-pyrido[1,2-a]pyrimidin-3-yl]butanamide

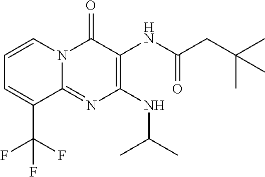 [SMILES: CC(C)Nc1nc2c(C(F)(F)F)cccn2c(=O)c1NC(=O)CC(C)(C)C]

Following the procedure of Example 6, Step 3, N-[2-chloro-4-oxo-9-(trifluoromethyl)-4H-pyrido[1,2-a]pyrimidin-3-yl]-3,3-dimethylbutanamide (0.50 g, 1.4 mmol, Example 6, Step 2) was reacted with isopropylamine (0.25 g, 4.2 mmol) to provide Example 7 (0.43 g, 80%).

¹H NMR (CDCl₃) δ (ppm) 8.94 (d, J=7.0 Hz, 1H), 7.92 (d, J=6.8 Hz, 1H), 7.33 (br, 1H), 6.93-6.87 (m, 1H), 6.35 (br s, 1H), 4.39-4.27 (m, 1H), 2.33 (s, 2H), 1.28 (d, J=6.4 Hz, 6H), 1.13 (s, 9H)

MS (ES+) 385 (ES−) 383

### Example 8

### 3,3-Dimethyl-N-[4-oxo-2-(propan-2-ylamino)-9-(trifluoromethyl)-6,7,8,9-tetrahydro-4H-pyrido[1,2-a]pyrimidin-3-yl]butanamide

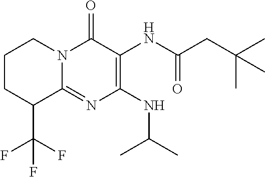 [SMILES: CC(C)Nc1nc2n(c(=O)c1NC(=O)CC(C)(C)C)CCCC2C(F)(F)F]

3,3-Dimethyl-N-[4-oxo-2-(propan-2-ylamino)-9-(trifluoromethyl)-4H-pyrido[1,2-a]pyrimidin-3-yl]butanamide (0.60 mg, Example 7) and Johnson Mattey Pd catalyst A503032 (25 mg) in ethanol (2 mL) was hydrogenated at 15 psi for 16 h. The mixture was filtered through a pad of Celite, and the filtrate was concentrated. The individual enantiomers were isolated by supercritical fluid chromatography (Chiralpak AD-H, mobile phase 80:20 CO₂1 methanol).

¹H NMR (CDCl₃) δ (ppm) 7.53 (br s, 1H), 6.18 (br s, 1H), 4.27-3.80 (m, 3H), 3.60-3.45 (m, 1H), 2.30 (s, 2H), 2.23-1.13 (m, 10H), 1.09 (s, 9H)

MS (ES+) 389 (ES−) 387

### Example 8a

The earlier peak: [α]_{D}²⁰=−24° (c 0.71, methanol)

### Example 8b

The later peak: [α]_{D}²⁰=+18° (c 0.65, methanol)

### Example 9

### N-[9-(1,1-Difluoroethoxy)-4-oxo-2-(propan-2-ylamino)-4H-pyrido[1,2-a]pyrimidin-3-yl]-3,3-dimethylbutanamide

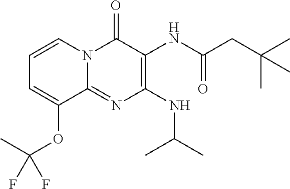 [SMILES: CC(C)Nc1nc2c(OC(C)(F)F)cccn2c(=O)c1NC(=O)CC(C)(C)C]

### Step 1. 3-(2-Bromo-1,1-difluoroethoxy)pyridin-2-amine

2-Amino-3-hydroxypyridine (730 mg, 6.6 mmol) and potassium hydroxide (409 mg, 7.6 mmol) were suspended in acetonitrile (30 mL) in an ice bath. 2-Bromo-1,1-difluoroethylene was bubbled slowly into the solution over 30 minutes. The reaction was allowed to warm to RT and stirred for 3 hours. The mixture was filtered through Celite; concentration in vacuo provided the titled compound (1.45 g, 87%) as a yellow solid.

¹H NMR (CDCl₃) δ (ppm) 7.96 (dd, J=5.0, 1.3 Hz, 1H), 7.39 (dd, J=7.9, 1.3 Hz, 1H), 6.64 (dd, J=7.9, 5.0 Hz, 1H), 4.80 (br, s, 2H), 3.82 (t, J=8.2 Hz, 2H)

MS (ES+) 253, 255

### Step 2. 3-(1,1-Difluoroethoxy)pyridin-2-amine

To a solution of 3-(2-bromo-1,1-difluoroethoxy)pyridin-2-amine (235 mg, 0.93 mmol) in ethanol (10 mL) was added 10% palladium on carbon (100 mg), and the reaction was hydrogenated at 45 psi (H₂) for 20 hours. The reaction was filtered to remove catalyst. Concentration of the filtrate afforded a white solid, which was partitioned between ethyl acetate and saturated aqueous sodium bicarbonate solution. The aqueous layer was extracted with ethyl acetate (2×25 mL). The combined organic layers were dried over MgSO₄, filtered, and concentrated to afford the titled compound (133 mg, 82%) as a solid.

¹H NMR (CDCl₃) δ (ppm) 7.91 (dd, J=5.1, 1.6 Hz, 1H), 7.38 (m, 1H), 6.64 (dd, J=7.9, 5.0 Hz, 1H), 4.66 (br, s, 2H), 1.96 (t, J=13.4 Hz, 3H)

MS (ES+) 175

### Step 3. 9-(1,1-Difluoroethoxy)-2-hydroxy-4H-pyrido[1,2-a]pyrimidin-4-one

Following the procedure of Example 4, Step 1, 3-(1,1-difluoroethoxy)pyridin-2-amine (3.1 g, 18 mmol) was converted to the titled compound (3.3 g, 77%).

¹H NMR (DMSO-d₆) δ (ppm) 11.80 (br s, 1H), 8.81 (d, J=6.8 Hz, 1H), 7.78 (d, J=7.6 Hz, 1H), 7.27-7.20 (m, 1H), 5.39 (br s, 1H), 2.06 (t, J=14.3 Hz, 3H)

MS (ES+) 243 (ES−) 241

### Step 4. 2-Chloro-9-(1,1-difluoroethoxy)-4H-pyrido[1,2-a]pyrimidin-4-one

Following the procedure of Example 1, Step 2, 9-(1,1-difluoroethoxy)-2-hydroxy-4H-pyrido[1,2-a]pyrimidin-4-one (4.2 g, 17 mmol) was converted to the titled compound (3.3 g, 73%).

¹H NMR (CDCl₃) δ (ppm) 8.88 (d, J=7.0 Hz, 1H), 7.76 (d, J=7.6 Hz, 1H), 7.19-7.12 (m, 1H), 6.50 (s, 1H), 2.10 (t, J=13.7 Hz, 3H)

MS (ES+) 261, 263

### Step 5. 2-Chloro-9-(1,1-difluoroethoxy)-3-nitro-4H-pyrido[1,2-a]pyrimidin-4-one

Following the procedure of Example 1, Step 3, 2-chloro-9-(1,1-difluoroethoxy)-4H-pyrido[1,2-a]pyrimidin-4-one (1.6 g, 6.1 mmol) was converted to the titled compound (1.6 g, 85%).

¹H NMR (CDCl₃) δ (ppm) 8.98 (d, J=7.0 Hz, 1H), 7.96 (d, J=7.8 Hz, 1H), 7.41-7.35 (m, 1H), 2.12 (t, J=13.9 Hz, 3H)

MS (ES+) 306, 308

### Step 6. 3-Amino-2-chloro-9-(1,1-difluoroethoxy)-4H-pyrido[1,2-a]pyrimidin-4-one

Following the procedure of Example 1, Step 4, 2-chloro-9-(1,1-difluoroethoxy)-3-nitro-4H-pyrido[1,2-a]pyrimidin-4-one (1.6 g, 5.2 mmol) was converted to the titled compound (1.34 g, 93%).

¹H NMR (CDCl₃) δ (ppm) 8.64 (d, J=7.2 Hz, 1H), 7.36 (d, J=7.2 Hz, 1H), 6.95 (dd, J=7.2 Hz, 1H), 2.08 (t, J=13.7 Hz, 3H)

MS (ES+) 276, 278

Step 7. N-[2-Chloro-9-(1,1-difluoroethoxy)-4-oxo-4H-pyrido[1,2-a]pyrimidin-3-yl]-3,3-dimethylbutanamide

Following the procedure of Example 1, Step 5, 3-amino-2-chloro-9-(1,1-difluoroethoxy)-4H-pyrido[1,2-a]pyrimidin-4-one (1.34 g, 4.9 mmol) was converted to the titled compound (1.41 g, 77.6%).

¹H NMR (CDCl₃) δ (ppm) 8.82 (d, J=7.0 Hz, 1H), 7.71 (d, J=7.6 Hz, 1H), 7.17-7.11 (m, 1H), 6.80 (br s, 1H), 2.30 (s, 2H), 2.09 (t, J=13.9 Hz, 3H), 1.14 (s, 9H)

MS (ES+) 374, 376

### Step 8

### Example 9

Following the procedure of Example 1, Step 6, N-[2-chloro-9-(1,1-difluoroethoxy)-4-oxo-4H-pyrido[1,2-a]pyrimidin-3-yl]-3,3-dimethylbutanamide (1.41 g, 3.77 mmol) was reacted with isopropylamine to afford Example 9 (1.07 g, 71.6%).

¹H NMR (CDCl₃) δ (ppm) 8.67 (d, J=6.8 Hz, 1H), 7.44 (d, J=7.4 Hz, 1H), 7.41 (br, 1H), 6.84-6.75 (m, 1H), 6.31 (br s, 1H), 4.39-4.35 (m, 1H), 2.31 (s, 2H), 1.99 (t, J=13.3 Hz, 3H), 1.27 (d, J=6.3 Hz, 6H), 1.10 (s, 9H)

MS (ES+) 397

### Example 10

### N-[2-Cyclopropyl-9-(1,1-difluoroethoxy)-4-oxo-4H-pyrido[1,2-a]pyrimidin-3-yl]-3,3-dimethylbutanamide

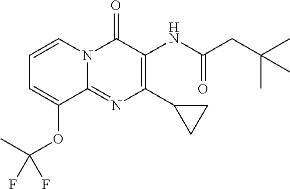 [SMILES: CC(C)(C)CC(=O)Nc1c(C2CC2)nc2c(OC(C)(F)F)cccn2c1=O]

### Step 1. 2-Cyclopropyl-9-(1,1-difluoroethoxy)-4H-pyrido[1,2-a]pyrimidin-4-one

Following the procedure of Example 4, Step 3, 2-chloro-9-(1,1-difluoroethoxy)-4H-pyrido[1,2-a]pyrimidin-4-one (1.7 g, 6.5 mmol, Example 9, Step 4) was converted to the titled compound (1.3 g, 75%).

¹H NMR (CDCl₃) δ (ppm) 8.83 (d, J=7.2 Hz, 1H), 7.54 (d, J=7.2 Hz, 1H), 6.94 (dd, J=7.2 Hz, 1H), 6.36 (s, 1H), 2.02 (t, J=13.5 Hz, 3H), 1.98-1.91 (m, 1H), 1.20-1.14 (m, 2H), 1.05-0.98 (m, 2H)

MS (ES+) 267

### Step 2. 2-Cyclopropyl-9-(1,1-difluoroethoxy)-3-nitro-4H-pyrido[1,2-a]pyrimidin-4-one

Following the procedure of Example 1, Step 3, 2-cyclopropyl-9-(1,1-difluoroethoxy)-4H-pyrido[1,2-a]pyrimidin-4-one (1.3 g, 4.9 mmol) was converted to the titled compound (1.4 g, 92%).

¹H NMR (CDCl₃) δ (ppm) 8.90 (d, J=7.2 Hz, 1H), 7.72 (d, J=7.4 Hz, 1H), 7.17-7.11 (m, 1H), 2.32-2.24 (m, 1H), 2.03 (t, J=13.5 Hz, 3H), 1.43-1.37 (m, 2H), 1.23-1.16 (m, 2H)

MS (ES+) 312

### Step 3. 3-Amino-2-cyclopropyl-9-(1,1-difluoroethoxy)-4H-pyrido[1,2-a]pyrimidin-4-one

Following the procedure of Example 1, Step 4, 2-cyclopropyl-9-(1,1-difluoroethoxy)-3-nitro-4H-pyrido[1,2-a]pyrimidin-4-one (1.4 g, 4.5 mmol) was converted to the titled compound (1.23 g, 97%).

¹H NMR (CDCl₃) δ (ppm) 8.66 (d, J=7.4 Hz, 1H), 7.23 (d, J=6.9 Hz, 1H), 6.86-6.81 (m, 1H), 4.10 (br s, 2H), 2.00 (t, J=13.5 Hz, 3H), 2.00-1.90 (m, 1H), 1.23-1.15 (m, 2H), 1.06-0.98 (m, 2H)

MS (ES+) 282

### Step 4

### Example 10

Following the procedure of Example 1, Step 5, 3-amino-2-cyclopropyl-9-(1,1-difluoroethoxy)-4H-pyrido[1,2-a]pyrimidin-4-one (1.23 g, 4.37 mmol) was converted to the titled compound (877 mg, 52.9%).

¹H NMR (CDCl₃) δ (ppm) 8.72 (d, J=6.8 Hz, 1H), 7.48 (d, J=7.4 Hz, 1H), 7.01 (br s, 1H), 6.96-6.89 (m, 1H), 2.33 (s, 2H), 2.16-2.06 (m, 1H), 1.99 (t, J=13.5 Hz, 3H), 1.31-1.24 (m, 2H), 1.14 (s, 9H), 1.09-1.02 (m, 2H)

MS (ES+) 380

### Example 11

### N-{2-[(2R)-Butan-2-ylamino]-9-methyl-4-oxo-6,7,8,9-tetrahydro-4H-pyrido[1,2-a]pyrimidin-3-yl}-3,3-dimethylbutanamide

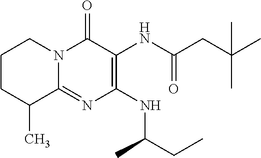 [SMILES: CC[C@@H](C)Nc1nc2n(c(=O)c1NC(=O)CC(C)(C)C)CCCC2C]

### Step 1. N-(2-Hydroxy-9-methyl-4-oxo-6,7,8,9-tetrahydro-4H-pyrido[1,2-a]pyrimidin-3-yl)-3,3-dimethylbutanamide

Diethyl[(3,3-dimethylbutanoyl)amino]propanedioate (3.7 g, 13.5 mmol, step 1a) was added to a suspension of 3-methylpiperidin-2-imine, hydrochloride salt (2.0 g, 13.3 mmol, step 1b) and sodium tert-butoxide (1.3 g, 13.5 mmol) in 3-methyl-1-butanol (3.4 mL). The reaction was irradiated for 60 minutes at 200° C. in a Biotage Initiator microwave system. The crude mixture was diluted with ethyl acetate, adsorbed onto Celite, and purified by silica gel column chromatography (0-3% methanol in ethyl acetate, gradient) to give the titled compound (1.2 g, 29%) as a thick orange oil.

¹H NMR (400 MHz, CDCl₃) δ ppm 13.03 (br, s, 1H), 8.06 (br s, 1H), 3.85-4.04 (m, 2H), 2.91 (m, 1H), 2.32 (s, 2H), 1.86-2.09 (m, 3H), 1.54-1.63 (m, 1H), 1.42 (d, J=7.0 Hz, 3H), 1.09 (s, 9H)

MS (ES+) 294 (ES−) 292

### Step 1a. Diethyl[(3,3-dimethylbutanoyl)amino]propanedioate

Triethylamine (75.5 mL, 542 mmol) was added to a rapidly stirred suspension of diethyl aminopropanedioate hydrochloride (39.0 g, 180 mmol) in dichloromethane (300 mL). The suspension was cooled to 0° C. and 3,3-dimethylbutanoyl chloride (26.0 mL, 180 mmol) was rapidly added drop-wise. The clear reaction mixture was warmed to RT and stirred for 18 hours. It was then added to water and washed with dichloromethane. The organic layer was washed with a 10% aqueous citric acid solution and with water, dried over MgSO₄, filtered, and concentrated in vacuo to give the titled compound (47.1 g, 96%) as a yellow oil.

¹H NMR (400 MHz, CDCl₃) δ ppm 6.41 (br d, J=6.4 Hz, 1H), 5.15 (d, J=6.8 Hz, 1H), 4.19-4.32 (m, 4H), 2.15 (s, 2H), 1.28 (t, J=7.1 Hz, 6H), 1.04 (s, 9H)

### Step 1b. 3-Methylpiperidin-2-imine, Hydrochloride Salt

3-Methylpyridin-2-amine (10 g, 90 mmol) was dissolved in ethanol (90 mL) containing concentrated hydrochloric acid (10 mL). Platinum oxide (2.1 g, 9.0 mmol) was added and the suspension was placed under 40 psi of hydrogen for 6.5 hours. The catalyst was removed by filtration through Celite. The filtrate was concentrated in vacuo to give the titled compound (14.4 g, 100%) as an off-white solid.

¹H NMR (400 MHz, CD₃OD) δ ppm 3.32-3.43 (m, 2H), 2.75-2.85 (m, 1H), 1.75-2.04 (m, 3H), 1.58-1.67 (m, 1H), 1.37 (d, J=7.4 Hz, 3H)

### Step 2. N-(2-Chloro-9-methyl-4-oxo-6,7,8,9-tetrahydro-4H-pyrido[1,2-a]pyrimidin-3-yl)-3,3-dimethylbutanamide

A mixture of N-(2-hydroxy-9-methyl-4-oxo-6,7,8,9-tetrahydro-4H-pyrido[1,2-a]pyrimidin-3-yl)-3,3-dimethylbutanamide (3.4 g, 12.0 mmol) and phosphorus oxychloride (21.4 mL, 232 mmol) was stirred at 70° C. for 2 hours. The mixture was dropped into stirring water containing sodium dihydrogen phosphate and occasionally cooled in an ice-water bath. The solution was washed with ethyl acetate. The organic extracts were washed with brine, dried over Na₂SO₄, filtered, and concentrated in vacuo. The crude material was purified by silica gel column chromatography (50-100% ethyl acetate in heptanes, gradient) to give the titled compound (1.9 g, 54%) as a light yellow foam.

¹H NMR (400 MHz, CDCl₃) δ ppm 6.85 (br s, 1H), 3.98-4.06 (m, 1H), 3.85-3.93 (m, 1H), 2.91-3.00 (m, 1H), 2.28 (s, 2H), 1.88-2.11 (m, 3H), 1.55-1.65 (m, 1H), 1.43 (d, J=7.0 Hz, 3H), 1.13 (s, 9H)

MS (ES+) 312, 314

### Step 3

### Example 11

A mixture of N-(2-chloro-9-methyl-4-oxo-6,7,8,9-tetrahydro-4H-pyrido[1,2-a]pyrimidin-3-yl)-3,3-dimethylbutanamide (1.0 g, 3.2 mmol), (R)-(−)-2-aminobutane (1.0 mL, 9.6 mmol) and diisopropylethylamine (2.3 mL, 12.8 mmol) in 3-methyl-1-butanol (2.5 ml) was irradiated for 30 min at 180° C. The reaction mixture was diluted with ethyl acetate, then washed with water and with brine. The organic layer was dried over Na₂SO₄, filtered, and concentrated in vacuo. The crude material was purified by silica gel column chromatography (0-5% methanol in ethyl acetate, gradient) to give Example 11 (1.09 g, 97%) as an off-white solid.

¹H NMR (400 MHz, CD₃OD) δ ppm 4.14-4.22 (m, 1H), 3.79-3.96 (m, 2H), 2.83-2.93 (m, 1H), 2.29 (s, 2H), 1.85-2.12 (m, 3H), 1.49-1.59 (m, 3H), 1.40 (d, J=7.0 Hz, 3H), 1.17 (d, J=6.4 Hz, 3H), 1.12 (s, 9H), 0.92 (t, J=7.4 Hz, 3H)

MS (ES+) 349 (ES−) 347

The diastereomeric products were isolated by supercritical fluid chromatography (Chiralpak AD-H column, 80:20 CO₂/ethanol mobile phase)

### Example 11a

Earlier-eluting peak: [α]_{D}²⁰=−50° (c 0.011, methanol)

### Example 11b

Later-eluting peak: [α]_{D}²⁰=−3° (c 0.011, methanol)

1. A compound of Formula I: 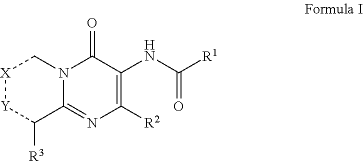
wherein:
R¹ is alkyl, cycloalkyl, wherein alkyl or cycloalkyl may be substituted with one or more halogen, alkoxy, aryl, or aryloxy;
R² is cycloalkyl or NR⁴R⁵;
R³ is H, halogen, alkyl, or alkoxy, wherein any alkyl may be substituted with one or more halogen atoms;
R⁴ and R⁵ are independently selected from H, C₁₋₆alkyl, C₃₋₆cycloalkyl, or —C₁₋₆alkyl-C₃₋₆cycloalkyl, wherein each alkyl or each cycloalkyl may be substituted with one or more halogen, provided that both R⁴ and R⁵ are not H simultaneously;
or R⁴ and R⁵, together with the N atom to which they are attached, form a heterocycloalkyl;
—X—Y— is ═CH—CH═, —CH₂—CH₂—, or —CH₂—; or a pharmaceutically acceptable salt thereof.

2. The compound of claim 1, wherein:
R¹ is C₅₋₆ alkyl;
R² is NR⁴R⁵, wherein one of R⁴ or R⁵ is H and the other is C₃₋₆alkyl, C₃₋₆cycloalkyl, or —C₁₋₃alkyl-C₃₋₆cycloalkyl;
R³ is halogen, alkyl, or alkoxy, wherein any alkyl may be substituted with one or more halogen atoms;
—X—Y— is ═CH—CH═ or —CH₂—CH₂—; or a pharmaceutically acceptable salt thereof.

3. The compound of claim 1, wherein:
R¹ is —CH₂C(CH₃)₃;
R³ is chloro, methyl, trifluoromethyl or 1,1-difluoroethoxy;
R⁴ is H and R⁵ is isopropyl, isobutyl, 1-cyclopropylethyl, cyclobutyl or cyclopentyl;
—X—Y— is ═CH—CH═ or —CH₂—CH₂—; or a pharmaceutically acceptable salt thereof.

4. The compound of claim 1, wherein:
R¹ is C₅₋₆ alkyl;
R² is C₃₋₆cycloalkyl;
R³ is halogen, or alkyl, wherein alkyl may be substituted with one or more halogen atoms;
—X—Y— is ═CH—CH═ or —CH₂—CH₂—; or a pharmaceutically acceptable salt thereof.

5. The compound of claim 1,
R¹ is —CH₂C(CH₃)₃;
R² is cyclopropyl;
R³ is chloro, methyl, trifluoromethyl or 1,1-difluoroethoxy;
—X—Y— is ═CH—CH═ or —CH₂—CH₂—; or a pharmaceutically acceptable salt thereof.

6. The compound of claim 1, wherein the compound is any example presented herein.

7. A method for the treatment of a disease, disorder or condition in a mammal comprising:
administering to said mammal in need thereof a therapeutically effective amount of a compound of claim 1 or a pharmaceutically acceptable salt thereof, where the disease, disorder, or condition is affected by the dampening of neuronal excitability characterized by the dysregulation of neuronal excitability in mammalian of the KCNQ family potassium ion channels.

8. The method or use of claim 7, wherein the KCNQ channel is a KV7.2/KV7.3 channels.

9. The method of claim 7, wherein the compound of Formula I or a pharmaceutically acceptable salt thereof is used to treat cognitive disorders, pain disorders, and epilepsy.

10. The method of claim 7, wherein the compound of Formula I or a pharmaceutically acceptable salt thereof is used to treat depression, including depression in cancer patients, depression in Parkinson's patients, post-myocardial Infarction depression, depression in patients with human immunodeficiency virus (HIV), Subsyndromal Symptomatic depression, depression in infertile women, pediatric depression, major depression, single episode depression, recurrent depression, child abuse induced depression, post partum depression, DSM-IV major depression, treatment-refractory major depression, severe depression, psychotic depression, post-stroke depression, neuropathic pain, manic depressive illness, including manic depressive illness with mixed episodes and manic depressive illness with depressive episodes, seasonal affective disorder, bipolar depression BP I, bipolar depression BP II, or major depression with dysthymia; dysthymia; phobias, including agoraphobia, social phobia or simple phobias; eating disorders, including anorexia nervosa or bulimia nervosa; chemical dependencies, including addictions to alcohol, cocaine, amphetamine and other psychostimulants, morphine, heroin and other opioid agonists, Phenobarbital and other barbiturates, nicotine, diazepam, benzodiazepines and other psychoactive substances; Parkinson's diseases, including dementia in Parkinson's disease, neuroleptic-induced parkinsonism or tardive dyskinesias; headache, including headache associated with vascular disorders; withdrawal syndrome; age-associated learning and mental disorders; apathy; bipolar disorder; chronic fatigue syndrome; chronic or acute stress; conduct disorder; cyclothymic disorder; somatoform disorders such as somatization disorder, conversion disorder, pain disorder, hypochondriasis, body dysmorphic disorder, undifferentiated disorder, and somatoform NOS; incontinence; inhalation disorders; intoxication disorders; mania; oppositional defiant disorder; peripheral neuropathy; post-traumatic stress disorder; late luteal phase dysphoric disorder; specific developmental disorders; SSRI “poop out” syndrome, or a patient's failure to maintain a satisfactory response to SSRI therapy after an initial period of satisfactory response; and tic disorders including Tourette's disease.

11. The method of claim 7, wherein the compound of Formula I or a pharmaceutically acceptable salt thereof is used in combination with another active pharmaceutical ingredient.
